(19) United States
(12) Patent Application Publication
(10) Pub. No.: US 2015/0165427 A1
(43) Pub. Date: Jun. 18, 2015

(54) METAL-MODIFIED ZEOLITE FOR CATALYTIC CRACKING OF HEAVY OILS AND PROCESS FOR PRODUCING LIGHT OLEFINS

(71) Applicant: King Fahd University of Petroleum and Minerals, Dhahran (SA)

(72) Inventors: Omar Ratib AWAYSSA, Dhahran (SA); Nabil Al-Yassir, Dhahran (SA); Abdullah Mohammed Aitani, Dhahran (SA); Sulaiman Saleh Al-Khattaf, Dhahran (SA)

(73) Assignee: King Fahd University of Petroleum and Minerals, Dhahran (SA)

(21) Appl. No.: 14/106,295

(22) Filed: Dec. 13, 2013

(51) Int. Cl. B01J 29/80; C07C 4/06

(52) U.S. Cl. CPC B01J 29/80; C07C 4/06

(57) ABSTRACT

The present invention relates to a fluid catalytic cracking (FCC) catalyst containing a metal-modified ZSM-5 zeolite catalyst additive for catalytically cracking heavy oils. The metal-modified ZSM-5 zeolite may be obtained by first preparing a mixture in which the metal is introduced to the zeolite via an impregnation method or an ion exchange method, stirring and drying the mixture, and then calcining to form the final metal-modified ZSM-5 zeolite. The metal-modified ZSM-5 zeolite is used with a FCC base catalyst at different ratios to increase the production of light olefins, particularly propylene, from the catalytic cracking of heavy oils.

### BACKGROUND

1. Technical Field

The present invention relates to a fluid catalytic cracking (FCC) process for improving the production of light olefins, especially ethylene, propylene, and n-butenes, from the cracking of hydrocarbons. More specifically, the invention relates to a metal-modified zeolite catalyst additive and its use for increasing the yield of light olefins from cracking heavy oils in an FCC process.

2. Description of the Related Art

The FCC process has been an important conversion process in petroleum refineries since its first commercial use. It is widely used to covert high-boiling, high molecular weight hydrocarbons to high value gasoline and light olefins, such as ethylene and propylene. Propylene has been widely used for downstream petrochemical applications. A rapid increase in world demand for propylene is driven by demand of various propylene derivatives, mainly polypropylene and propylene oxide. While 30% of the world's 80 million tons/year of propylene is supplied by refinery FCC operations, 57% is co-produced from steam cracking of naphtha or other feedstocks, and the remaining 13% is produced by on-purpose processes such as propane dehydrogenation, olefin metathesis, high-severity FCC and other methods.

ZSM-5 zeolite has been added to FCC base catalyst to maximize yields of propylene. Using ZSM-5 zeolite as an FCC catalyst additive is an efficient and cost-effective method for improving propylene yield because it offers refiners a high degree of flexibility to their FCC units. As pointed out by Wallenstein et al. in “The Dependence of ZSM-5 Additive Performance on the Hydrogen-transfer Activity of the RE-USY Base Catalyst in Fluid Catalytic Cracking,” Appl. Catal. A, 2001, 214: 11-29 (incorporated herein by reference), restriction of hydrogen transfer reaction is one of the important measures in increasing the yield of propylene over ZSM-5 zeolite. Indeed, the utilization of ZSM-5 zeolite as a separate additive to FCC catalysts is known since the late 1980s for enhancing gasoline octane number.

However, the increase in yields of propylene with increasing concentration of ZSM-5 zeolite is achieved at the cost of gasoline yield. For example, Adewuyi et al. describes in “Effects of High-level Additions of ZSM-5 to a Fluid Catalytic Cracking (FCC) RE-USY catalyst,” Appl. Catal. A, Gen 1995, 131: 121-133 (incorporated herein by reference), that ZSM-5 zeolite additive mixed with an FCC base catalyst cracks gasoline range olefins (C4-C12) into liquefied petroleum gas (LPG) (C3-C4 olefins) thereby decreasing the overall gasoline yield.

Nevertheless, as reported by Knight et al. in “Maximize Propylene in Your FCC Unit,” Hydrocarbon Processing, 2011, 90(9): 91-95 (incorporated herein by reference), the use of ZSM-5 zeolite as FCC catalyst additive has increased steadily since early 1990s to a level where it is currently used in almost 20% of the worldwide FCC's 14 million barrel/day capacity units. In some commercial FCC units, the amount of the ZSM-5 crystal added to FCC base catalysts has reached as high as 25-30 wt %.

In addition, modifying ZSM-5 zeolite with other elements, such as transitional metals of Fe, Ti, Ni, Zr, and Mn, has received increasing attention in various catalytic applications including cracking of hydrocarbons because of the catalytic properties of the transitional metals in addition to the catalytic properties of ZSM-5 zeolite such as acidity and shape-selectivity. Transition metal-modified ZSM-5 zeolite helps in the conversion of heavier molecules and overcomes the loss in activity of FCC catalysts.

Li et al. reported in “Interaction of Titanium and Iron Oxide with ZSM-5 to Tune the Catalytic Cracking of Hydrocarbons,” App. Catal. A: Gen. 2010, 375: 222-229 (incorporated herein by reference), that the combined modification of ZSM-5 zeolite with Fe₂O₃ and TiO₂ shows better performance towards light olefins production than Ti/ZSM-5, Fe/ZSM-5 or unmodified ZSM-5 in the cracking of n-decane and isopropyl benzene.

U.S. Patent Application Publication No. US 2009/0134065 to Cheng et al. (incorporated herein by reference) also describes a ZSM-5 catalyst for enhancing light olefin production in FCC process comprising 5.0 wt % P₂O₅ and 1.0 wt % Fe₂O₃ outside of the zeolite's framework. The catalyst is disclosed to be highly active as compared to other catalysts and to show a high selectivity for propylene.

U.S. Patent Application Publication No. US 2009/0124842 to Reagan et al. (incorporated herein by reference) discloses an improved cracking catalyst comprising a large pore zeolite and a medium or small pore zeolite for the production of propylene from a hydrocarbon feedstock. The medium or small pore catalyst includes a metal (selected from the group consisting of Ga, Cu, Zn, Ge, Cd and their mixtures) deposited on the medium or small pore catalyst.

U.S. Pat. No. 7,531,706 to Wakui et al. (incorporated herein by reference) discloses a catalytic cracking process for producing light olefins such as ethylene and propylene from gaseous or liquid hydrocarbons using pentasil type zeolite catalyst modified with rare earth elements and at least Mn or Zr.

U.S. Patent Application Publication No. US 2011/0039688 to Choi et al. (incorporated herein by reference) describes a similar approach by employing MnO and P₂O₅ modified ZSM-5 zeolite for the catalytic cracking of naphtha to light olefins in a severe operating condition of high temperatures and high humidity.

U.S. Patent Application Publication No. 2011/0270009 to de Souza et al. (incorporated herein by reference) describes a process for maximizing the production of light olefins, preferably ethylene, by the catalytic cracking of C4-C6 saturated hydrocarbons using Ni-ZSM-5 catalyst. The concentration of NiO was in the range of 0.1-20 wt % and the cracking temperature was in the range of 400-650° C.

### BRIEF SUMMARY

One objective of the present invention is to provide a metal-modified ZSM-5 zeolite catalyst to be used as a catalyst additive for cracking heavy oils. Another objective is to modify the ZSM-5 zeolite so as to obtain a zeolite catalyst which can increase the propylene yield at a less expense of the gasoline yield. Another objective is to provide a simple method for preparing the metal-modified ZSM-5 zeolite. Another objective is to provide a process for producing light olefins from heavy oils using the metal-modified ZSM-5 zeolite catalyst in an FCC unit. These and other objectives have been achieved according to the present invention.

In one embodiment, the metal-modified ZSM-5 zeolite is a ZSM-5 zeolite comprising at least one metal selected from the group consisting of Be, Mg, Ca, Sr, Ba, Ra, Ti, Cr, Mn, Fe, Co, Ni, Cu, Zn, and Zr.

In another embodiment, the metal-modified ZSM-5 zeolite is a Mn-modified ZSM-5 zeolite.

In another embodiment, the metal-modified ZSM-5 zeolite is one free from rare earth elements.

In another embodiment, the metal-modified ZSM-5 zeolite is one free from phosphorous.

In another embodiment, the metal-modified ZSM-5 zeolite catalyst is used as an FCC catalyst additive.

In another embodiment, the metal-modified ZSM-5 zeolite catalyst is used to improve the yield of light olefins, such as ethylene, propylene, and n-butenes.

In yet another embodiment, the metal-modified ZSM-5 zeolite is used to produce light olefins from the catalytic cracking of heavy oils.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF THE DRAWING(S)

A more complete appreciation of the disclosure and many of the attendant advantages thereof will be readily obtained as the same becomes better understood by reference to the following detailed description when considered in connection with the accompanying drawings, wherein:

FIG. 1 is a schematic illustration of a procedure for preparing a metal-modified ZSM-5 zeolite of the present invention.

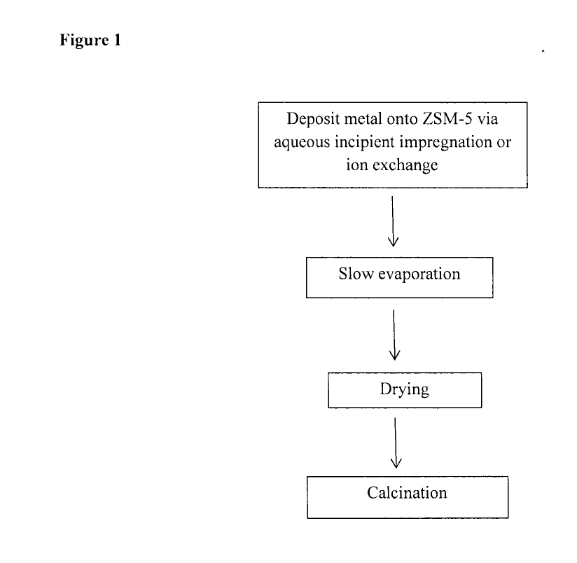

### DETAILED DESCRIPTION

The particular style of the catalytic cracking reaction of the present invention is not restricted. The reaction may be carried out by supplying a high-boiling feedstock of long-chain hydrocarbon molecules to a reactor loaded with hot FCC catalyst, which is a find powder. The particle size distribution of each of the FCC catalyst and the additive ranges between 10 and 200 μm, and more preferably between 20 and 150 μm. The average particle size ranges between 40 and 100 μm, and more preferably between 60 and 80 μm. The reactor is not particularly specified. It can be a fixed bed reactor, a moving bed reactor, or a fluidized bed reactor. The hydrocarbon feedstock is vaporized and cracked into small molecules by contacting with the hot catalytic layer in the reactor at a cracking temperature ranging from 400-700° C., more preferably 500-600° C.

The reaction product vapors are further separated, based on various boiling points, into gaseous products, gasoline, light gas oil, heavy gas oil, slurry oil, etc. After each use, the catalyst may be reheated to burn off the coke to obtain a regenerated catalyst. The heat generated by the combustion of the coke is partially absorbed by the regenerated catalyst and provides the heat required for the vaporization of the feedstock and the endothermic cracking reactions.

Catalysts possessing properties such as good stability at high temperature, high activity, large pore sizes, good resistance to attrition, and low coke production, are particularly suitable as an FCC catalyst. The zeolite used as an FCC catalyst is composed of silica and alumina tetrahedra with each tetrahedron having either an aluminum or a silicon atom at the center and four oxygen atoms at the corners. The distinctive lattice structure of zeolite thus functions as a molecular sieve that allows only a certain size range of molecules to enter the lattice. In particular, Y-type zeolites, such as zeolite Y and ultrastable Y-type (USY) zeolite, and ZSM-5 zeolites are widely used catalysts in an FCC process.

ZSM-5 zeolite is a zeolite mineral belongs to the pentasil family of zeolites. Its chemical formula is NaₙAlₙSi₉₆₋ₙO₁₉₂.16H₂O (0<n<27). ZSM-5 zeolite is formed largely of 5-member rings. Those 5-member rings are connected to each other and organized as columns to form channels, particularly the straight channels along the [0 1 0] direction and the sinusoidal channels along the [1 0 0] direction. These channels further intersect and form two- or three-dimensional channel systems so that particles can diffuse within the structure. ZSM-5 zeolite is a versatile solid-acid catalyst and the broad range of ZSM-5 zeolite compositions is one reason for its catalytic versatility. It is possible to prepare ZSM-5 with Si/Al ratios from about 8 to infinity. In addition, it is possible to prepare ZSM-5-based materials with other elements incorporated into the framework.

The present invention provides a simple method for modifying ZSM-5 zeolite with a transition metal and/or an alkaline earth metal (hereafter referred to as the “metal-modified ZSM-5 zeolite”). For example, the metal-modified ZSM-5 zeolite may comprise at least one metal selected from the group consisting of Be, Mg, Ca, Sr, Ba, Ra, Ti, Cr, Mn, Fe, Co, Ni, Cu, Zn, and Zr. The metal-modified ZSM-5 zeolite may be used as a separate FCC catalyst additive to improve the yield of light olefins, such as ethylene and propylene, from catalytic cracking of heavy oils.

In the present invention, the metal-modified ZSM-5 zeolite has a Si/Al molar ratio in the range of 25-800, preferably 30-500, and more preferably 80-300. The incorporation of metal into the ZSM-5 zeolite framework can be achieved by any of the known methods such as impregnation or ion exchange. The metal loading of the metal-modified ZSM-5 zeolite is 0.1-15 wt %, preferably 0.5-10 wt %, and more preferably 1.0-5.0 wt %. The metal-modified ZSM-5 zeolite can be used as a catalyst additive and blended with a base catalyst, such as a zeolite Y and a USY zeolite, in various concentrations from 0.1 to 30 wt %, preferably from 0.5 to 28 wt %, and more preferably from 1.0 to 25 wt %, wherein the wt % is a weight percentage based on the total weight of the metal-modified ZSM-5 zeolite and the base catalyst. The concentration of the metal-modified ZSM-5 zeolite additive should not exceed 30 wt %, because an excessive concentration of the additive has the effect of dilution of the FCC base catalyst.

The FCC base catalyst is preferably a large-pore USY zeolite. The base catalyst may also contain other components such as a matrix, a binder, and fillers, which may be used to improve catalyst performance and provide physical strength and integrity of the catalyst. The matrix may contain materials such as amorphous alumina. The binder may contain silica sol. The fillers may contain clay and other materials, such as silica, alumina, magnesia or quartz sand.

The average pore diameter of the USY FCC base catalyst ranges from 5-500 Å, preferably 10-250 Å, 20-100 Å, 40-80 Å, or 50-60 Å. The USY FCC catalyst has a surface area of 50-500 m²/g, preferably 75-400 m²/g, 100-300 m²/g, or 200-250 m²/g; and a pore volume of 0.05-0.50 cm³/g, preferably 0.1-0.45 cm³/g, 0.15-0.40 cm³/g, or 0.2-0.35 cm³/g. Preferably, before use, the base catalyst is calcined at a temperature of 300-800° C., preferably 400-600° C., for several hours.

The metal-modified ZSM-5 zeolite exhibits surprisingly excellent selectivity in the cracking of gasoline-range olefins to lighter olefins, especially propylene and n-butenes, when it is blended with a FCC base catalyst in cracking heavy oils such as vacuum gas oil (VGO). The feed oil used in the invention is heavy-fraction oil preferably with a boiling point higher than 250° C. at the atmospheric pressure and having hydrocarbons with carbon atoms of more than 20. The heavy oil may include straight-run gas oil, VGO, atmospheric residue, or vacuum residue. These aforementioned heavy-fraction oils may be used alone or as a mixture.

The catalyst/oil ratios for the FCC process of the present invention range from 0.5-10 g/g, preferably 1.0-8 g/g, more preferably 1.5-5.0 g/g, or 2.0-4.0 g/g.

A Mn-modified ZSM-5 zeolite is described next in detail as an example of the metal-modified ZSM-5 zeolite of the present invention. ZSM-5 zeolite can be modified with Mn-containing precursor salts such as manganese nitrate or sulfate. Manganese can be deposited by any of the known methods such as impregnation or ion exchange. The amount of Mn to modify ZSM-5 can be in the range of 0.1-15.0 wt %, preferably 0.5-10 wt %, and more preferably 1.0-5 wt %, wherein the wt % is a weight percentage based on the total weight of the ZSM-5 zeolite. Preferably, prior to ZSM-5 modification with the Mn-containing precursor, the ZSM-5 zeolite is dried in the presence of air. Further drying can be achieved by heating at a temperature of 60-150° C., preferably 80-100° C., for 6-20 hours, preferably 8-12 hours. The obtained Mn-containing ZSM-5 zeolite is then calcined at a temperature ranging from 400-650° C., preferably from 500-600° C., to transform the precursor salt to manganese oxide. Finally, the calcination is finished in air at a heating rate of 2-8° C./min, preferably 2.5-5° C./min, more preferably 3° C./min, with holding time of 2-8 hours, preferably 4-6 hours, more preferably 5 hours, to form the final Mn-modified ZSM-5 zeolite.

Modifying ZSM-5 zeolite with manganese provides a catalyst composition with dehydrogenation function in addition to ZSM-5 acid sites and pore selectivity. Therefore, using the Mn-modified ZSM-5 zeolite as a separate FCC catalyst additive can significantly improve light olefin yields. Propylene yield may double as a result of blending the Mn-modified ZSM-5 zeolite with a FCC base catalyst in the cracking of VGO. The improvement in propylene yield can be attributed to the presence of a cluster of nanostructured MnO₂ particles. Moreover, the partial reduction in the pore diameter of ZSM-5 micropores and thus the reduction in the extent of hydrogen transfer reactions improve the yield of light olefins at FCC cracking conditions. Therefore, manganese modification provides dehydrogenating sites that are capable of dehydrogenating larger paraffins into C5+ olefins that can be further cracked into light olefins.

Additionally, the decrease in the gasoline yield when the Mn-modified ZSM-5 zeolite catalyst additive is used is less than the decrease in the gasoline yield when an unmodified ZSM-5 zeolite additive is used.

The present invention is further explained in the following examples with the understanding that this invention is not restricted to these examples.

### EXAMPLES

The catalytic performance of the metal-modified ZSM-5 zeolite was evaluated in a fixed-bed microactivity test (MAT) according to the ASTM method. The feedstock was hydrotreated VGO with properties as shown in Table 1. All MAT runs were performed at a cracking temperature of 550° C. and a time-on-stream of 30 s. Conversion was varied by changing catalyst/oil (C/O) ratio in the range of 1.5 to 5.0 g/g. The commercial equilibrium FCC catalyst (E-Cat) was based on USY zeolite with a surface area of 135 m²/g and a pore volume of 0.23 cm³/g. This E-Cat was calcined at 500° C. for 3.0 hours before further use. The unmodified and Mn-modified ZSM-5 zeolites were added at 25 wt % with E-Cat prior to MAT evaluation.

TABLE 1

| | Value |
|---|---|
| Property | |
| Density (g/cm³) (15° C.) | 0.896 |
| Sulfur (ppm) | 300 |
| Nitrogen (ppm) | 170 |
| Saturates (wt %) | 59 |
| Aromatics (wt %) | 40 |
| Residue (wt %) | 0.8 |
| Simulated Distillation (° C.) | |
| Initial boiling point | 308 |
| 5% | 348 |
| 25% | 376 |
| 50% | 420 |
| 90% | 507 |
| Final boiling point | 568 |

Gaseous products (dry gas and LPG) were analyzed using two gas chromatographs equipped with FID/TCD detectors. Coke on catalyst was determined by a carbon analyzer. For liquid products, three different cuts were considered: gasoline (C5, <221° C.), LCO (light cycle oil, 221-343° C.), and HCO (heavy cycle oil, >343° C.). The weight percentage of liquid products was determined by a simulated distillation gas chromatography (GC) according to ASTM D-2887. MAT conversion was defined as the sum of yields for dry gas (H₂ and C1-C2), LPG (C3-C4), gasoline, and coke.

### Example 1

1.0 g of HZSM-5 (from Zeolyst) with a Si/Al molar ratio=30 was stirred in 10 ml aqueous solution containing 0.10 g of manganese salt precursor (manganese (II) nitrate hexahydrate) that corresponded to a Mn loading of 2.0 wt %. The mixture was stirred for 3 h and then the solvent was removed by slow evaporation at 60° C. in a drying oven. The product was dried at 100° C. overnight and then calcined at 550° C. at a heating rate of 3° C./min with holding time of 5 h thereby forming additive A. The same procedure was repeated to form additive B by using 0.20 g of manganese (II) nitrate hexahydrate that corresponded to a Mn loading of 4.0 wt %. The MAT performance results at constant conversion (70%) for VGO cracking over the base E-Cat, E-Cat/unmodified ZSM-5(30), and E-Cat/additives (A and B) are presented in Table 2.

TABLE 2

| | | E-Cat/25% Additive | | |
|---|---|---|---|---|
| | E-Cat | ZSM-5(30) | Additive A | Additive B |
| Mn loading, wt % | 0 | 0 | 2.0 | 4.0 |
| Catalyst/oil ratio, g/g | 1.7 | 2.5 | 1.7 | 1.8 |
| Product yield, % | | | | |
| Dry Gas | 3.2 | 5.8 | 4.6 | 4.7 |
| H₂ | 0.05 | 0.2 | 0.05 | 0.08 |
| C₁ | 0.7 | 0.9 | 0.7 | 0.8 |
| C₂═ | 1.4 | 3.3 | 3.0 | 2.9 |
| C₂ | 0.8 | 1.3 | 0.9 | 0.9 |
| LPG | 15.9 | 31.0 | 32.8 | 32.4 |
| C₃═ | 5.0 | 7.8 | 12.6 | 13.0 |
| C3 | 0.6 | 8.6 | 2.0 | 1.7 |
| C₄═ | 7.4 | 6.5 | 13.2 | 13.1 |
| n-C₄ | 0.4 | 2.5 | 1.2 | 0.9 |
| i-C₄ | 2.1 | 5.3 | 3.7 | 3.2 |
| C₂═-C₄═ | 13.8 | 17.3 | 28.9 | 28.9 |
| Gasoline | 50.0 | 33.5 | 32.3 | 32.4 |
| LCO | 12.8 | 8.1 | 10.9 | 11.5 |
| HCO | 17.2 | 21.8 | 19.1 | 18.6 |
| Coke | 0.5 | 0.8. | 0.20 | 0.6 |
| C3═/Gasoline ^{a} | Base | 3.4 | 8.7 | 9.1 |

^{a} percent increase in propylene yield per unit decrease in gasoline yield

### Example 2

The procedure of Example 1 was repeated by using HZSM-5 (from Zeolyst) with a Si/Al ratio=80 to form additives C and D with 2.0 wt % and 4.0 wt % Mn loadings, respectively. The MAT performance results at constant conversion (70%) for VGO cracking over the base E-Cat, E-Cat/unmodified ZSM-5(80), and E-Cat/additives (C and D) are presented in Table 3.

TABLE 3

| | | E-Cat/25% Additive | | |
|---|---|---|---|---|
| | E-Cat | ZSM-5(80) | Additive C | Additive D |
| Mn loading, wt % | 0 | 0 | 2.0 | 4.0 |
| Catalyst/oil ratio, g/g | 1.7 | 1.8 | 2.3 | 2.0 |
| Product yield, % | | | | |
| Dry Gas | 3.2 | 5.1 | 5.4 | 4.4 |
| H₂ | 0.05 | 0.07 | 0.1 | 0.08 |
| C₁ | 0.7 | 0.8 | 0.9 | 0.8 |
| C₂═ | 1.4 | 3.2 | 3.3 | 2.5 |
| C₂ | 0.8 | 1.0 | 1.0 | 0.9 |
| LPG | 15.9 | 31.4 | 33.3 | 29.2 |
| C₃═ | 5.0 | 10.7 | 13.1 | 11.4 |
| C3 | 0.6 | 3.8 | 2.35 | 1.6 |
| C₄═ | 7.4 | 10.6 | 12.6 | 11.9 |
| n-C₄ | 0.4 | 1.7 | 1.2 | 0.8 |
| i-C₄ | 2.1 | 4.8 | 4.2 | 3.4 |
| C₂═-C₄═ | 13.8 | 24.5 | 29.0 | 25.9 |
| Gasoline | 50.0 | 33.4 | 30.8 | 36.0 |
| LCO | 12.8 | 11.2 | 10.8 | 11.3 |
| HCO | 17.2 | 19.0 | 19.1 | 18.4 |
| Coke | 0.5 | 0.8 | 0.8 | 0.7 |
| C3═/Gasoline ^{a} | Base | 6.9 | 8.4 | 9.1 |

^{a} percent increase in propylene yield per unit decrease in gasoline yield

### Example 3

The procedure of Example 1 was repeated by using HZSM-5 (from Zeolyst) with a Si/Al ratio=280 to form additives E and F with 2.0 wt % and 4.0 wt % Mn loadings, respectively. The MAT performance results at constant conversion (70%) for VGO cracking over the base E-Cat, E-Cat/unmodified ZSM-5(280), and E-Cat/additives (E and F) are presented in Table 4.

TABLE 4

| | | E-Cat/25% Additive | | |
|---|---|---|---|---|
| | E-Cat | ZSM-5(280) | Additive E | Additive F |
| Mn loading, wt % | 0 | 0 | 2.0 | 4.0 |
| Catalyst/oil ratio, g/g | 1.7 | 2.3 | 2.0 | 1.8 |
| Product yield, % | | | | |
| Dry Gas | 3.2 | 4.6 | 4.0 | 3.7 |
| H₂ | 0.05 | 0.06 | 0.06 | 0.06 |
| C₁ | 0.7 | 0.8 | 0.8 | 0.7 |
| C₂═ | 1.4 | 2.8 | 2.3 | 2.0 |
| C₂ | 0.8 | 0.9 | 0.9 | 0.8 |
| LPG | 15.9 | 29.8 | 31.1 | 28.9 |
| C₃═ | 5.0 | 11.7 | 12.6 | 11.6 |
| C3 | 0.6 | 1.9 | 1.4 | 1.2 |
| C₄═ | 7.40 | 11.5 | 13.1 | 12.4 |
| n-C₄ | 0.4 | 0.9 | 0.7 | 0.7 |
| i-C₄ | 2.1 | 3.9 | 3.3 | 3.0 |
| C₂═-C₄═ | 13.8 | 25.9 | 26.0 | 26.0 |
| Gasoline | 50.0 | 35.0 | 37.1 | 37.1 |
| LCO | 12.8 | 11.4 | 12.1 | 12.1 |
| HCO | 17.2 | 18.6 | 17.9 | 17.9 |
| Coke | 0.5 | 0.6 | 0.5 | 0.5 |
| C3═/Gasoline ^{a} | Base | 8.9 | 11.8 | 10.2 |

^{a} percent increase in propylene yield per unit decrease in gasoline yield

The MAT results of Examples 1 to 3 show the advantages of the addition of Mn-modified ZSM-5 zeolites to the FCC base catalyst, as evident by improvement in propylene yield in all examples with the addition of the Mn-modified ZSM-5 zeolites regardless of the Si/Al ratio of the starting ZSM-5 zeolites. The percent increase in propylene yield per unit decrease in gasoline yield was above 8% for all Mn-modified ZSM-5 zeolite additives (A to F) as compared with an increase of 3.4% for the unmodified ZSM-5 zeolite additive. This improvement in propylene yield in the Mn-modified ZSM-5 zeolite additives can be attributed to the presence of Mn on the surface of the zeolite which promoted the formation of reactive unsaturated hydrocarbons, favoring the production of light olefins, especially ethylene, propylene, and n-butenes.

The MAT results as shown in Tables 2-4 show that VGO cracking activity of E-Cat/25 wt % Mn-modified ZSM-5 zeolite additives did not decrease and the highest propylene yield of 13.1 wt % was achieved over E-Cat/additive C (Si/Al ratio=80 and Mn loading=2.0 wt %, and conversion=70%) as compared with propylene yield of 10.7 wt % over unmodified ZSM-5 zeolite additive. The yield of butenes also increased to 12.6 wt % from 10.6 wt % over E-Cat/Mn-modified ZSM-5(80) additive. Table 5 shows comparative C3=yields for all additives at a constant C/O ratio of 3. The maximum C3=yield was found for additive C (14.8 wt %) as compared with other additives.

TABLE 5

| Catalyst | Additive | C3═ yield, wt. % |
|---|---|---|
| E-Cat | — | 7.1 |
| E-Cat/ZSM-5(30) | ZSM-5(30) | 8.2 |
| E-Cat/ZSM-5(80) | ZSM-5(80) | 11.7 |
| E-Cat/ZSM-5(280) | ZSM-5(280) | 13.2 |
| E-Cat/A (Example 1) | 2 wt. % Mn-ZSM-5(30) | 13.2 |
| E-Cat/B (Example 1) | 4 wt. % Mn-ZSM-5(30) | 14.1 |
| E-Cat/C (Example 2) | 2 wt. % Mn-ZSM-5(80) | 14.8 |
| E-Cat/D (Example 2) | 4 wt. % Mn-ZSM-5(80) | 13.5 |
| E-Cat/E (Example 3) | 2 wt. % Mn-ZSM-5(280) | 14.1 |
| E-Cat/F (Example 3) | 4 wt. % Mn-ZSM-5(280) | 13.1 |

The foregoing discussion discloses and describes merely exemplary embodiments of the present invention. As will be understood by those skilled in the art, the present invention may be embodied in other specific forms without departing from the spirit or essential characteristics thereof. Accordingly, the disclosure of the present invention is intended to be illustrative, but not limiting of the scope of the invention, as well as other claims. The disclosure, including any readily discernible variants of the teachings herein, defines, in part, the scope of the foregoing claim terminology such that no inventive subject matter is dedicated to the public.

What is claimed is:

1. A fluid catalytic cracking catalyst, comprising:
a metal-modified ZSM-5 zeolite as a catalyst additive; and
a zeolite base catalyst other than a ZSM-5 zeolite,
wherein:
a metal loading of the metal-modified ZSM-5 zeolite is from 0.1 to 15 wt %, based on a total weight of the metal-modified ZSM-5 zeolite; and
the metal-modified ZSM-5 zeolite is present in an amount of from 1 to 25 wt % based on a total weight of the catalyst.

2. The catalyst of claim 1, wherein the metal-modified ZSM-5 zeolite has a Si/Al molar ratio of from 25 to 800.

3. The catalyst of claim 1, wherein the zeolite base catalyst is a USY zeolite.

4. The catalyst of claim 1, wherein the metal-modified ZSM-5 zeolite comprises at least one metal selected from the group consisting of Be, Mg, Ca, Sr, Ba, Ra, Ti, Cr, Mn, Fe, Co, Ni, Cu, Zn, and Zr.

5. The catalyst of claim 1, wherein the metal-modified ZSM-5 zeolite is a Mn-modified ZSM-5 zeolite.

6. The catalyst of claim 5, wherein the Mn-modified ZSM-5 comprises Mn in an amount of from 1 to 5 wt %, based on a total weight of the Mn-modified ZSM-5 zeolite.

7. The catalyst of claim 1, wherein the metal-modified ZSM-5 zeolite is free from rare earth element and phosphorous.

8. A process for preparing a metal-modified ZSM-5 zeolite, the process comprising:
obtaining a metal-comprising ZSM-5 zeolite;
drying the metal-comprising ZSM-5 zeolite, thereby obtaining a dried ZSM-5 zeolite; and
calcining the dried ZSM-5, thereby obtaining the metal-modified ZSM-5 zeolite.

9. The process of claim 8, wherein said obtaining is carried out by
impregnating a ZSM-5 zeolite with a solution comprising a metal precursor to obtain the metal-comprising ZSM-5 zeolite, or
adding a ZSM-5 zeolite to a solution comprising a metal precursor to obtain the metal-comprising ZSM-5 zeolite by ion exchange.

10. The process of claim 9, wherein the metal-modified ZSM-5 zeolite is a Mn-modified ZSM-5 zeolite.

11. The process of claim 10, wherein the metal precursor comprises a manganese-comprising compound.

12. The process of claim 11, wherein the manganese-comprising compound is at least one selected from the group consisting of manganese nitrate and manganese sulfate.

13. A process for producing a light olefin from a heavy oil, the process comprising:
cracking the heavy oil in the presence of a catalyst, thereby obtaining the light olefin,
wherein:
the light olefin is at least one of ethylene, propylene, and a n-butene;
the heavy oil comprises more than 20 carbon atoms and is at least one selected from the group consisting of a straight-run gas oil, a VGO, an atmospheric residue, and a vacuum residue;
the catalyst comprises a metal-modified ZSM-5 zeolite, and a zeolite base catalyst other than a ZSM-5 zeolite;
a metal loading of the metal-modified ZSM-5 zeolite is from 0.1 to 15 wt %, based on a total weight of the metal-modified ZSM-5 zeolite; and
the metal-modified ZSM-5 zeolite is present in an amount of from 1.0 to 25 wt % based on a total weight of the catalyst.

14. The process of claim 13, wherein the zeolite base catalyst is a USY zeolite.

15. The process of claim 13, wherein the metal-modified ZSM-5 zeolite is a Mn-modified ZSM-5 zeolite.

16. The process of claim 13, wherein the light olefin is propylene.

17. The process of claim 13, wherein the catalyst consists of the Mn-modified ZSM-5 zeolite and the zeolite base catalyst.
